(19) United States
(12) Patent Application Publication
(10) Pub. No.: US 2026/0298081 A1
(43) Pub. Date: Oct. 1, 2026

(54) METHODS AND DEVICES FOR GENERATING FLOW THROUGH POROUS MEDIA USING DRILL CUTTINGS

(71) Applicant: UNIVERSITY OF WYOMING, Laramie, WY (US)

(72) Inventors: Amin AMOOIE, Windsor, CO (US); Bradley MCCASKILL, Laramie, WY (US); Mohammad SEDGHI, Laramie, WY (US); Yanbin GONG, Laramie, WY (US); Mohammad PIRI, Laramie, WY (US)

(21) Appl. No.: 19/479,286

(22) PCT Filed: Apr. 26, 2024
(86) PCT No.: PCT/US2024/026443

§ 371 Date: Oct. 27, 2025

Related U.S. Application Data
(60) Provisional application No. 63/462,664, filed on Apr. 28, 2023.

(51) Int. Cl. E21B 49/00; G01N 15/08; G01N 33/24

(52) U.S. Cl. CPC E21B 49/005; G01N 15/0826; G01N 33/241

(57) ABSTRACT

A method and system for generating flow through porous media using drill cuttings are disclosed. The system and methods include obtaining a set of raw images of one or more drill cuttings from the porous media sample. A set of processed images are generated based on the set of raw images. A representative network of pores and throats are constructed using the set of processed images. A fluid flow through the representative network is predicted.

### BACKGROUND

### Field

Aspects of the present disclosure generally relate to generating flow through porous media, and more particularly, to capture fluid flow through a porous medium represented by drill cuttings.

### Description of the Related Art

Modeling techniques for fluid flow through porous media are broadly implemented for petroleum resource development, materials engineering, food packaging, and medical technology development. Fluid flow modeling techniques may be equipped to illustrate both physical and chemical media properties like permeability, capillary pressure, fluid saturation, contact angle, wettability, or other similar properties, which may be used to characterize fluid behavior.

Although current techniques for modeling fluid flow through porous media are based on technological advancements made over many years, resultant models may still be tenuous representations of actual porous media. For example, fluid flow models of porous media exceeding a few millimeters may require a lower resolution implementation to match currently available computational capabilities. As a result, fluid flow models based on porous media of a larger scale may not accurately reflect physical and chemical properties of the media. Accordingly, there is an impetus to improve the accuracy of fluid flow modeling, including, for example: improving image processing techniques to allow for higher resolution model input and model output, improving image processing techniques to allow for more accurate model input and model output, enhancing computational processing capability to reduce computational expense, enhancing computational processing capability increase modeling speed, increasing automation for iterative modeling steps, improving model capability for dynamic modeling of different fluid flow environments, improving model capability for dynamic modeling of larger fluid flow environments, and the like.

Consequently, there exists a need for further improvements in fluid flow modeling of porous media to overcome the aforementioned technical challenges and other challenges not mentioned.

### SUMMARY

One aspect of the present disclosure provides a method for image processing by one or more processing units. The method may include obtaining a set of raw images of one or more drill cuttings from the porous media sample. A set of processed images are generated based on the set of raw images. A representative network of pores and throats are constructed using the set of processed images. A fluid flow through the representative network is predicted.

One aspect provides a non-transitory computer-readable medium comprising computer-executable instructions that, when executed by one or more processors, cause one or more CPUs to perform a method of image processing. The method may include obtaining a set of raw images of one or more drill cuttings from the porous media sample. A set of processed images are generated based on the set of raw images. A representative network of pores and throats are constructed using the set of processed images. A fluid flow through the representative network is predicted.

Another aspect provides an apparatus for processing a porous media sample comprising a memory and one or more processors. The one or more processors are configured to cause the apparatus to obtaining a set of raw images of one or more drill cuttings from the porous media sample. A set of processed images are generated based on the set of raw images. A representative network of pores and throats are constructed using the set of processed images. A fluid flow through the representative network is predicted.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only example aspects and are therefore not to be considered limiting of its scope, may admit to other equally effective aspects.

FIG. 1A depicts a ball and stick representation of an example pore network overlaid with the segmented micro-CT image of a porous media (sandstone rock sample) that it was extracted from, according to embodiments.

FIG. 1B depicts an example high-resolution porous media images taken by a scanning instrument from a single rock sample and segmented for characterization, according to embodiments.

FIG. 2 depicts an example core-flooding instrument for determining the physical and chemical characteristics of a porous media sample, according to embodiments.

FIG. 3 depicts example drill cuttings from a porous media sample, according to embodiments.

FIGS. 4A and 4B is a flow diagram illustrating an example pore network extraction procedure, according to embodiments.

FIG. 5 is a flow diagram illustrating an example pore network extraction procedure, according to embodiments.

FIG. 6 is a flow diagram illustrating an example pore network modeling procedure, according to embodiments.

FIG. 7 is a flow diagram illustrating an example pore network modeling procedure, according to embodiments.

FIG. 8 is a flow diagram illustrating certain operations by one or more processing units, according to embodiments.

FIG. 9 is an example device for imaging of porous media, according to embodiments.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

In the following, reference is made to aspects of the disclosure. However, it should be understood that the disclosure is not limited to specifically aspects described. Instead, any combination of the following features and elements, whether related to different aspects or not, is contemplated to implement and practice the disclosure. Furthermore, although aspects of the disclosure may achieve advantages over other possible solutions and/or over the prior art, whether or not a particular advantage is achieved by a given aspect is not limiting of the disclosure. Thus, the following aspects, features, aspects, and advantages are merely illustrative and are not considered elements or limitations of the appended claims except where explicitly recited in a claim(s). Likewise, a reference to “the disclosure” shall not be construed as a generalization of any inventive subject matter disclosed herein and shall not be considered to be an element or limitation of the appended claims except where explicitly recited in a claim(s).

The present disclosure relates to techniques for high-reliability image processing of porous media. Specifically, the techniques discussed herein may be implemented for use in processing accurate, high-resolution images of porous media to produce reliable digital replicates of any porous medium. The porous media may comprise a rock sample, a core sample, a plastic sample, a tissue sample, or any other organic or inorganic sample having pore space ascertainable through imaging techniques.

A thorough grasp of fluid flow through pore networks of material with substantial pore space may be consequential to enhancing technical efficacy of fluid flow techniques in a wide range of industries. Models of fluid flow through a porous material may be used to describe physical and chemical characteristic of the material. Such models may help to highlight optimal usage for a certain porous material. In many cases, networks of pores within a material are extremely small, typically in the order of micros. Techniques for characterizing these pore networks are hindered by the computational expense of modeling at a microscale. Often, to alleviate computational burdens, pore-network modeling techniques use lower resolution images at the expense of model accuracy. Extrapolation errors caused by low resolution characterization may result in mischaracterization of physical and chemical characteristics of the porous material. Accordingly, ideal modeling of fluid flow through porous media would allow for rapid, accurate microscale imaging that may be performed without inhibitive computational expense.

According to certain aspects of the present disclosure, high-reliability image processing of porous material may be achieved through multi-image analysis performed by processing systems operating in parallel. Specifically, overlapping high-resolution images of a porous material, may be obtained by a scanning instrument (e.g., a micro-computed tomography (CT) scanner), stitched together where the images overlap, and processed to obtain a super-resolution image of the porous material. Stitching procedures may be split across one or more central processing units (CPUs) and/or one or more graphics processing units (GPUs) in a parallel processing architecture. This may allow faster, more accurate modeling of porosity information without loss of vital micro-porosity detail.

Implementation of techniques for obtaining reliable digital replicates of porous media samples as described herein may enhance pore-network modeling functionality by reducing porous material characterization errors to the benefit of all users seeking a more comprehensive understanding of any given porous material.

### Introduction to Pore-Network Modeling

Modeling techniques for fluid flow through porous media may illustrate both physical and chemical porous media properties. Models of porous media may be used to ascertain permeability, capillary pressure, fluid saturation, wettability, buoyancy, and the like to a greater degree of accuracy comparable to physical flooding of a porous media sample. Additionally, physical and chemical properties determined using pore network modeling techniques may be used to characterize in-situ fluid behavior as it travels through the porous media under a wide variety of wettability and flooding conditions. These conditions may not be accessible to users performing conventional physical flooding characterization techniques.

Permeability is the tendency of the porous media to allow liquids to flow through it. Capillary pressure is the pressure difference existing across the interface separating two immiscible fluids. Fluid saturation is the measurement of fluid present in the pore spaces of the porous media. Contact angle is a measured angle between a fluid-fluid or a fluid-gas interface at a point where it meets a solid surface. Wettability is the ability of a liquid to maintain contact with a solid surface. Wettability may vary depending on wettability conditions and the type of wetting liquid present in the porous media sample. For example, a water-wet medium may show a lower wetting affinity to the oil phase than an oil-wet medium, where higher or lower wetting is determined with respect to a given phase. In certain cases, the correlation between wettability and viscosity ratio may not be straightforward, as there may be water or oil wet conditions with similar viscosities.

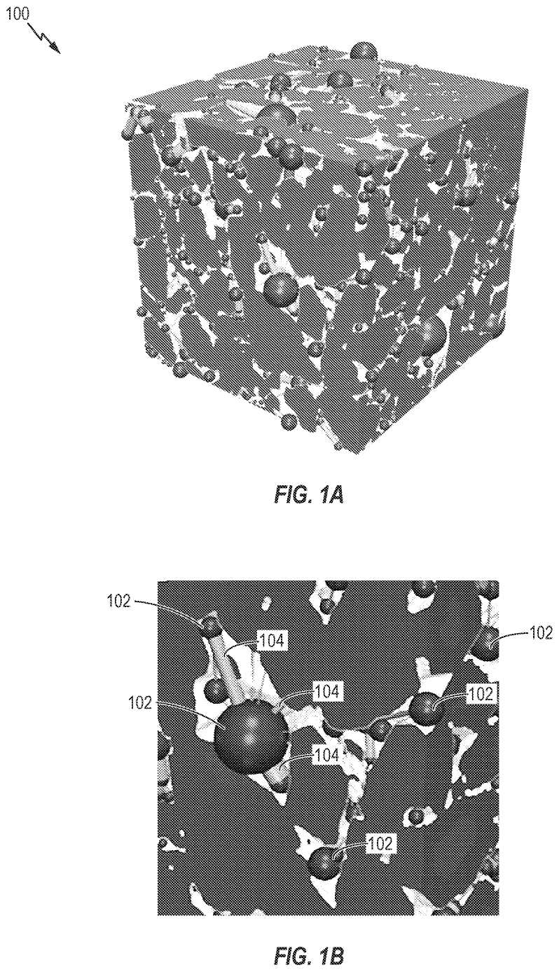

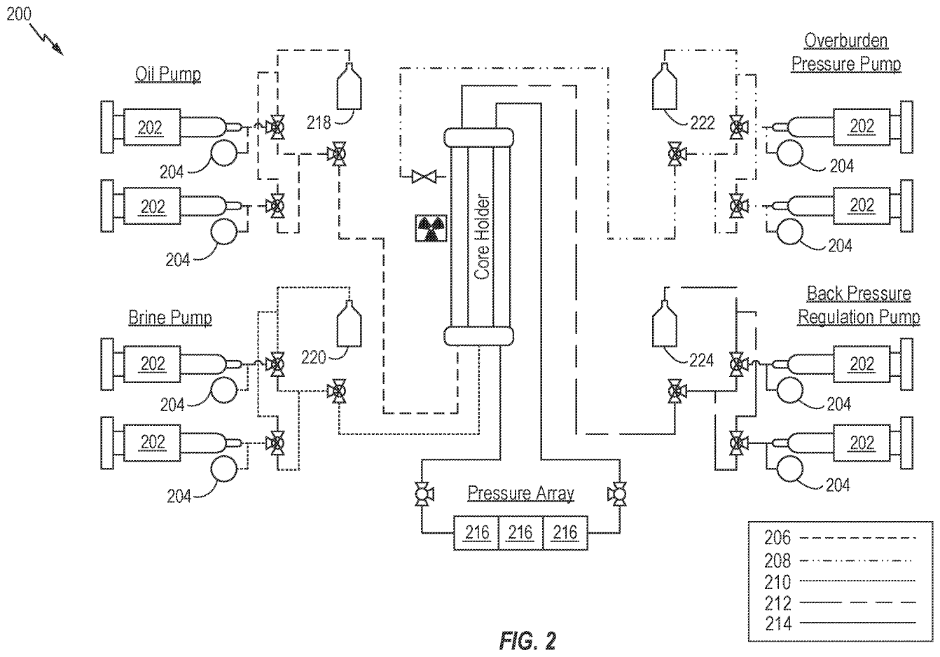

A modeled pore network is a practical description of a porous medium targeted for fluid flow modeling. FIG. 1A illustrates an example section of a pore network extracted from porous sandstone. The section of the pore network describes the porosities of various size and shape present in that portion of the sandstone and may be used to model fluid flow through those porosities for various wettability conditions. Three-dimensional (3D) portions of a pore network model may more accurately characterize the porous media sample either alone or in combination with other 3D portions of the pore network model.

Dynamic pore network models may be used to model multi-phase fluid flow using physically based displacement mechanisms (PBDMs) across pores defined in a pore network (e.g., of FIG. 1A) extracted from images of a targeted porous medium. PBDMs may represent an estimated displacement of a modeled fluid in response to movement of another fluid or gas within the pore network. As immiscible phases react with one another throughout the pore network during fluid flooding, PBDMs are induced where, for example, capillary pressure across a meniscus exceeds the wettability constraints on either phase. Fluid saturation, contact angle, buoyancy, and the like may also affect PBDMs throughout a pore network. By utilizing a pore network model extracted from a porous media sample, a user may be able to ascertain PBDMs through the porous media sample under a wide variety of wettability conditions in order to ultimately obtain, for example, useful permeabilities for a larger sample of the porous medium without degrading a porous media sample via repeated physical flooding.

To properly generate PBDMs at a pore scale for the targeted porous media, imaging may capture complex geometries of the targeted porous media at a resolution sufficiently high to retain acceptable accuracy. An example of these geometries is illustrated in FIG. 1B. Pores may be defined as a complex polyhedron having at least a center 102 and spherical and effective diameters. Connective throats 104 between pores may also be defined. In many cases, image resolution may be in micrometers or nanometers to capture applicable pore detail. High-resolution pore models allow for accurate rendering of the fluid flow characteristics described above as ascertained at each pore and for each PBDM.

PBDMs may occur upon flooding or draining of a pore network model, where aqueous phase injection or removal is iteratively simulated through the pore network. Aqueous flooding and aqueous draining may be implemented in various modeled wettability conditions, where certain fluids are present prior to the start of a simulation. Wettability conditions may include at least water-wet, oil-wet, or mixed-wet conditions. During aqueous flooding, injected water may displace immiscible fluid preexisting in the pore network model. During aqueous draining, injected immiscible fluid may displace water preexisting in the pore network model. In certain cases, flooding and draining may be fluid flooding and fluid draining. In some cases, fluid may be oil.

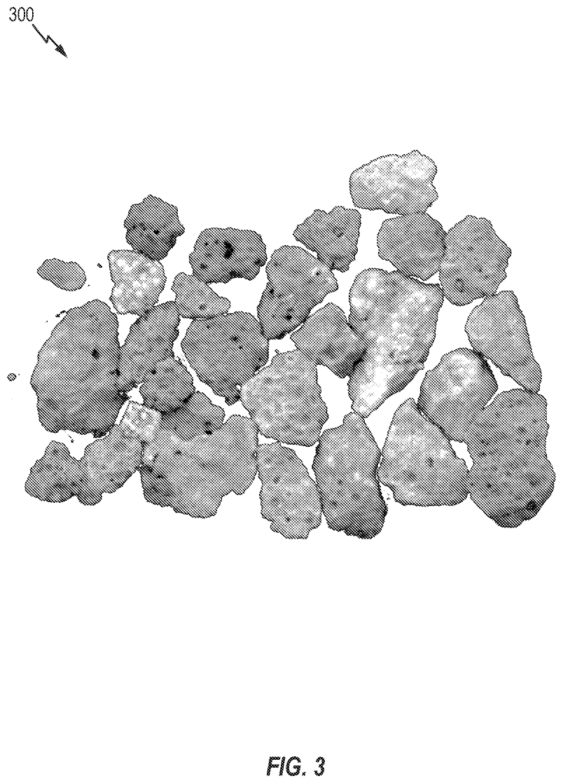

Flooding or draining of a pore network model may be simulated based in part on scanned images of physical flooding implemented by a flooding instrument 200 of FIG. 2. In some cases, a porous media may undergo a core-flooding experiment to establish an irreducible water saturation, a residual oil saturation, or both. Core-flooding may be enabled by a set of pumps 202, rupture disks 204, pump lines 206-214, differential pressure transducers 216, and source buckets 218-222 working in tandem to flood a porous media sample loaded in a core holder. In some cases, a scanning instrument (e.g., a micro computed tomography (micro-CT) scanner) captures a dry reference image prior to flooding. Scanning occurs in a field of view defined within the core holder. In some cases, the porous media sample may be flooded with brine from bucket 220 via the brine tubing line 206 and scanned again to ensure that the porous media sample is fully saturated. Once the brine flooding is complete, the absolute permeability of the porous media sample may be obtained. The oil flooding may be performed alongside additional brine flooding. Any fluid expelled as a result of overburden pressure (i.e., pressure that compacts pore space and reduces permeability) may be transported via the confining fluid line 208 and collected in bucket 222. Any fluid expelled as a result of the flooding procedure may be transported via the effluent fluid line 212 and collected in bucket 224. In many cases, core sample pressure may be iteratively adjusted during flooding. Pressure may be recorded by one or more differential pressure transducers 216 coupled to the core holder via a transducer line 214.

Scanned images obtained from flooding procedures performed by the flooding instrument 200 of FIG. 2 may be used to extract a pore map representative of the porous media sample. The images may be processed to determine characteristics of fluid flow through the porous media sample. In many cases, the images may also be used to extract a representative pore network model.

Imaging of porous media is typically performed using micro-CT imaging. In many cases, commercial micro-CT scanners (e.g., Zeiss scanners) are available for imaging necessary to perform pore-network modeling. Images of porous media taken by micro-CT scanners are at a sufficiently high resolution to create a microscale digital image of the porous media.

In the current state of the art, there exists a challenge of extracting porous media characteristics in a manner precise and repeatable to ensure the ultimate stability of future simulations. Currently, techniques for porous media characterization require lengthy step-wise processing known to incur undue computational expense and introduce instability to characterization of the porous media sample. As a result, users may not be able to rely on characterization output to simulate flow conditions in a useful way.

### Aspects Related to Capturing Fluid Flow through a Drill Cutting Sample

In the current state of the art, there exists a challenge of predicting fluid flow through porous media in a manner precise and repeatable to ensure the ultimate stability of future simulations. Currently, techniques for two-phase fluid flow prediction require lengthy step-wise processing known to incur undue computational expense and introduce instability to characterization of the porous media sample. As a result, users may not be able to rely on characterization output to simulate flow conditions in a useful way.

Fluid flow modelling through porous media is often utilized to enhance petroleum resource development. In recent years, global demand for energy resources has mobilized development of petroleum reservoirs as targets for hydrocarbon extraction. The geological formations that comprise these hydrocarbon reservoirs are ultra-tight shale formations resistant to primary petroleum extraction techniques. A matrix of an ultra-tight shale reservoir may be characterized by low permeability and low porosity. To extract hydrocarbons from the ultra-tight shale matrix, secondary and tertiary petroleum extraction techniques seek to maximize oil production through the microscale pore networks that comprise a substantial amount of the porosity in the shale matrix.

A robust understanding of fluid flow through microscale pore networks of unconventional reservoirs may be consequential to extracting the trillions of barrels of oil still housed in shale formations globally. Models of fluid flow through a pore network that describe permeability, capillary pressure, fluid saturation, and wettability may help to elucidate specific steps to be taken during resource development to optimize petroleum production. Even so, techniques for characterizing these microscale pore networks are hindered by the computational expense of modeling sub-resolution pore network and extrapolation errors caused by unstable characterization of pore geometries.

As discussed above, ideal modeling of fluid flow through porous media would allow for high resolution imaging of a macro-sized porous media sample. In a case where the porous media sample is, for example, a cylindrical core sample of a rock having a length of six inches and a diameter of one inch, the core sample is likely to have porosity and permeability that vary across its length and width. This is common in core samples, and especially in core samples representative of ultra-tight oil formations. Geological processes that form certain oil-bearing rocks can produce heterogeneous morphological features in the rock that may be present even at a nanometer scale. This is especially true for oil bearing carbonate rocks, which contain micro-porosities that contribute significantly to the overall porosity of the rock. These nanoscale morphological features may affect the pore network of the core sample, altering the porosity and permeability throughout a core sample. Thus, accurate characterization of fluid flow through a core sample may depend on high-resolution (e.g., microscale resolution, nanoscale resolution) sufficient to detect heterogeneous properties of a pore network.

Numerical computation from high-resolution 3D micro-tomographic images of rocks, also known as Digital Rock Technologies (DRT), has the potential to more accurately predict the underlying petrophysical properties and macroscopic flow behavior of fluids within such porous media. The first component of DRT is image processing of micro-CT images, which may entail processing of images obtained by micro-CT scanner in the lab. Ideally, DRT may accurately and efficiently render and process the obtained image datasets, which may allow a user to obtain reliable digital replicates that are in one-to-one correspondence to the rock samples. However, in the current state of the art, DRT is limited to samples with simple structure and mineralogy, and may have low computational efficiency, ultimately making DRT less than desirable to process large image datasets (high resolutions and/or large physical sample sizes). Additionally, the majority of existing attempts require continuous end-user input and interaction for each case. This prevents scaling out by preventing automated execution and batch processing of a number of cases. There also exists a challenge in processing multi-mineral rocks that are presented using grayscale values of different minerals in micro-CT images, because those values are often very similar.

In some cases, DRT may be applied to drill cuttings. Drill cuttings are pieces of rocks and a byproduct of drilling cores (e.g., from oil and gas reservoirs). These cuttings can vary in size, shape, and pore microstructure, depending on the depth at which they are gathered and the sedimentary facies from which they originated. The ability to identify the facies of drill cuttings is an important step in reservoir characterization and can provide valuable information about the nature and properties of subsurface lithology.

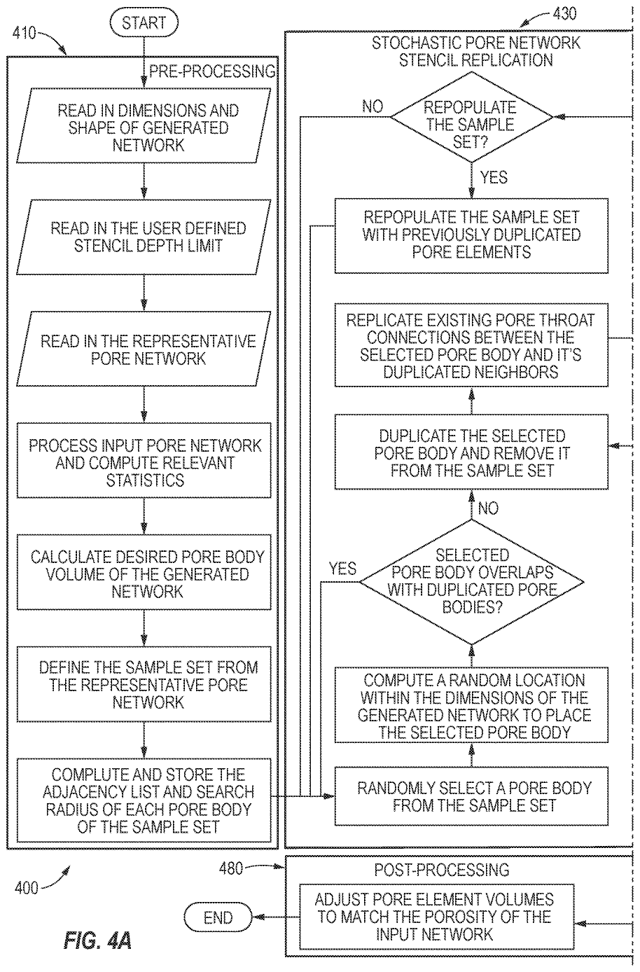

Example drill cuttings are illustrated in FIG. 3. Drill cuttings are fragments of a porous media sample (e.g., a rock sample) generated during well drilling. In situations where core output is low, such as when drilling through weakly cemented reservoirs with high porosity and permeability, collecting cutting samples becomes necessary. However, the information obtained from analyzing drill cuttings is limited. Generally, cutting analysis is used to supplement routine and special core analysis in order to acquire more detailed geological and petrophysical information when core data is unavailable. However, because the drill cutting are irregular and lack coordination with any specific geologic facies, information as available in the current state of the art is subject to error.

Having an efficient cuttings analysis technology may decrease drilling costs and time. As an example, partially replacing 75% of core drilling with reverse circulation (RC) drilling (which involves cuttings sampling) could lead to substantial financial and time savings.

There are numerous issues with utilizing drill cuttings in DRT. In one example, drill cuttings are typically small, irregularly shaped, and fragmented, which makes it difficult to obtain a representative volume or an accurate representation of the porous media microstructure. In another example, sample preparation may be difficult because proper sample preparation is crucial for DRP analysis. Cleaning, mounting, and polishing cuttings can be difficult due to their small size and irregular shape. This can lead to inaccuracies in imaging and simulations. In another example, high-resolution 3D imaging techniques like X-ray micro-CT are often used in DRT studies. However, drill cuttings may require even higher resolutions to capture the intricate pore structures accurately, which may be challenging and time-consuming. In another example, obtaining a representative sample from drill cuttings may be challenging, as they may not accurately represent the entire formation. The small size of cuttings can limit the ability to detect and analyze macroscopic features or variations in the porous media. In another example, geological formations may be highly heterogeneous, with varying mineralogy, porosity, and permeability. Accurately capturing this heterogeneity in a small, fragmented sample of drill cuttings can be difficult. In another example, the drilling process can cause mechanical damage to the cuttings, including crushing, smearing, or alteration of the original rock fabric. This can affect the accuracy of DRT analysis.

### Aspects Related to Image Processing

Aspects of the present disclosure provide techniques for image processing that utilize highly parallelized denoising and filtering techniques. Techniques described herein also provide an AI-assisted, CPU-parallelized, and GPU-accelerated automated segmentation workflow that combines adaptive local thresholding, watershed segmentation, and clustering techniques to seamlessly segment a variety of images into multiple phases and infer under-resolved regions of complex porous media.

In some cases, if any porous media Routine Core Analysis measurements are available, the processors may begin to implement a drill cutting characterization workflow after 3D micro-CT images of a selection of drill cuttings and available cores are obtained. Following pre-processing, filtering, and segmentation of the images from plugs, the one or more processors conduct comprehensive pore and grain structure analysis to calibrate the framework. In particular, the one or more processors may calibrate the image processing using a prediction of total porosity as compared to and calibrated against Routine Core Analysis measurements of various physical porous media. The detailed measurement of macro-and micro-porosity through the image-based inference of the under-resolved phase may predict a total porosity value for any or all of the provided drill cuttings. If such calibration is not warranted, the one or more processors may proceed with pre-processing the drill cuttings images and normalizing their gray-scale intensity, followed by the segmentation. In some cases, the processors may maintain certain model parameters for all drill cuttings.

The features mentioned above may provide key information on statistical distribution of pore microstructure for full-scale segmentation of the 3D image stack. In some cases, full-scale segmentation of the 3D image stack may be computationally expensive for a high number of drill cuttings. To alleviate this computational expense, the one or more processors may utilize gray-scale images of drill cuttings for feature extraction. Three approaches for utilizing gray-scale images of drill cuttings are proposed herein, but are not limiting of the scope, applicability, or aspects set forth in this disclosure.

A first approach uses GLCM and GLSZM, analysis, according to certain aspects of the present disclosure. Micro-CT images are 3D voxel-based representations of porous samples captured at a particular image resolution. The intensity of each voxel may correspond to the material encountered by X-rays, with low intensity representing pores or empty spaces, high intensity representing solid components (e.g., silicate, calcite, apatite, bauxite, plastics, etc.), and very-high intensity representing significantly dense components (e.g., iron oxides, pyrite, barite, other metals, plastics, etc.). The variation in intensity levels may be analyzed using three main procedures: histograms, GLCM, and GLSZM. These procedures are dimension reduction techniques that summarize a large amount of information into principal components, making them critical for analyzing data with high dimensions and complexity, such as porous media images.

Gray-level histograms are a preliminary dimension reduction technique that represent features of a porous structure as a frequency-based distribution. As the simplest representation for a 3D micro-CT image, histograms illustrate the dominant components identified as modes of the histogram distribution. In addition to histograms, techniques described herein may allow the one or more processors to apply pattern recognition and texture analysis techniques to supplement histogram information.

GLCM performed by one or more processors may produce spatial information by analyzing the neighborhood of a reference pixel using different angles and distance parameters, allowing for directional analysis at different scales. GLSZM is direction independent and may also produce spatial information of gray-level intensities by analyzing their connectivity in a 3D manner. In many cases, GLSZM procedures implemented by one or more processors may highlight both dominant and minor features. Dominant features may occur as peaks with high-to low-connectivity. Minor features may occur as medium-to low-connectivity peaks.

In some cases, the one or more processors may use GLCM representation to extract principal information about texture of each drill cutting sample. Using this information, the one or more processors may classify the dataset based on physical (e.g., mineralogical, petrophysical, lithological) heterogeneities associated with different facies. In particular, the second-order statistics from GLCM may be obtained to better understand porous media sample structure in terms of grain sizes, anisotropy, and heterogeneity. Furthermore, the dominant and minor features obtained by GLSZM may be used as analogs to porosity and permeability of drill cuttings. Such analogs allow the one or more processors to proceed without completely processing and segmenting the images and/or performing fluid flow procedures. Using these statistical analogs, the processors may also be able to infer grayscale representative elementary volumes (GREVs) of different drill cuttings of various sizes in an efficient and robust manner.

Once the one or more processors obtain trinary images from the raw drill cutting images, the one or more processors may perform instance segmentation. In some cases, the processors may perform instance segmentation via a 3D distance transform watershed procedure, which segments 3D pore space into labeled pore bodies separated by interfaces that represent the throats or constrictions within a pore region.

According to certain aspects of the present disclosure, one or more processors may use both PCA and auto-encoders to extract relevant features from the micro-CT 3D images of drill cuttings. These features can capture the underlying patterns and variations in the images that distinguish one facies from another. By using these features as part of the inputs feature space to a clustering algorithm, techniques described herein are able to group the drill cuttings into different clusters that correspond to different facies.

The number of principal components needed to capture the underlying structure of the data may vary depending on the complexity of the problem and the amount of variance illustrated for each component. While it is possible that one principal component is enough to capture the majority of the variability in the dataset, it is also possible that multiple principal components may better capture the variability in the dataset. The one or more processors may perform a scree plot analysis to determine the number of principal components to retain. A scree plot shows the eigenvalues of each principal component in decreasing order, and the platform automatically finds the point where the eigenvalues level off, indicating the point beyond which additional principal components do not contribute much to the variability in the data.

A third approach uses feature extraction. In a case where the one or more processors have obtained 3D image datasets for a number, N, of drill cuttings, the processors may implement certain steps to perform feature extraction. First, the one or more processors may perform a 3D multi-label semantic segmentation using a highly parallelized, AI-assisted marker-controlled compact watershed procedure. This procedure, which outputs a trinary image defining pores, clay (e.g., clays having micro-porosity), and grain regions. The processors may then perform instance segmentation of a binary image (e.g., a binary image containing a pore component and a second component represented as the sum of grain and clay components). The instance segmentation may be based on 3D distance transform watershed, and may be conducted to segment the 3D pore space into labeled pore bodies and throat interfaces.

In some cases, the processors perform 3D morphological region analysis to obtain key features, including one or more of at least total porosity of the specimen, effective pore radius, maximum inscribed ball radius, Euler characteristic number, sphericity and aspect ratio of pores. During this step, the processors may output a number of features equivalent to 6×N features.

Next, the one or more processors perform GLSM on gray-scale images for statistical texture analysis of drill cuttings. The processors may calculate several second-order statistical measures according to the following: (1) GLCM contrast describing local variation in the gray-level intensities, (2) GLCM angular second moment, describing the rock homogeneity; (3) GLCM mean, describing weighted average of the probability of occurrence of features based on their location on the GLCM map; and (4) GLCM correlation, measuring the linear dependencies of grayscale values and the degree of (an) isotropy in the micro-computed tomographic images of each of the rock types. During this step, the processors may output a number of features equivalent to 4×N features.

In some cases, the processors may proceed by implementing GLSZM to analyze gray-scale images of drill cuttings. The processors may calculate statistical descriptors of pore space to understand the connectivity of low gray-level intensities. Specifically, two statistical measures may be implemented to describe the pore space of the porous media sample: Low Gray-Level Emphasis (LGE) describing the entire pore-space region of low gray-levels and Large Area Low Gray-Level Emphasis (LALE) describing large, connected pore space of low gray levels. LGE may be understood as an analog to porosity, while LALE is associated with permeability. This step outputs 2×N features.

The processors may then extract features from the 3D gray-scale image dataset via PCA according to the following procedure: First, the processors reshape each 3D binary image stack into a 1D array. Next, the one or more processors apply PCA to one of the 1D arrays associated with one drill cutting, and perform the scree plot analysis to determine the number of principal components to retain, denoted as P. The processors then apply PCA to a number, N, 1D arrays, resulting in NXP principal components as features for clustering.

In some cases, the processors may use an auto-encoder to extract features from the 3D image dataset as follows: First, the one or more processors design an auto-encoder architecture that takes a 3D image stack as input and outputs a compressed representation of the image. The processors train the auto-encoder using the N image stacks, and extract the compressed representations of the N image stacks from the bottleneck layer of the trained auto-encoder. In some cases, the processors apply the GLCM, GLSZM, PCA and auto-encoders to gray-scale images, but it also allows their execution on binary segmented images. The former leads to extraction of features that better capture the overall shape and structure of the drill cuttings since gray-scale images may be more informative as they preserve the intensity information. Depending on the performance of grouping the cuttings into different facies, features from binary images may be added to, or replace, those extracted from the gray-scale images.

Aspects of the present disclosure seek to automate micro-CT analyses of drill cuttings and reservoir core plugs by pattern recognition and machine learning. Using advanced clustering algorithms, techniques described herein allow one or more processors to group the drill cuttings into different clusters based on the extracted features that can capture the underlying patterns and variations in the images as described above. If the number of clusters k, corresponding to the number of facies, is known a priori, for instance through geological interpretations and the knowledge of depth interval at which the cuttings were gathered, then a k-means clustering algorithm with k clusters may be selected. Next, the one or more processors can apply k-means clustering to the resulting feature matrix to group the drill cuttings into k facies based on their feature similarity, using the following workflow: First, the processors initialize the k-means algorithm with k cluster centroids and train the procedure on the feature matrix. Then, the processors assign each 3D image stack to the cluster with the nearest centroid and visualize the resulting clusters to see if they correspond to different facies.

If the number of facies is not known in advance, the number of clusters may be determined by evaluating the within-cluster sum of squares (inertia) under a number of k. The k where additional number of clusters do not substantially decrease the inertial is the optimal k. Silhouette score may also be implemented, which provides a measure of how well the data points fit into their assigned clusters. Once the number of clusters is determined, the processors implement a procedure to assign each sample to the nearest cluster center based on their feature vectors.

To complement the k-means clustering, a model-based clustering procedure, the one or more processors implement GMM, which is used to automatically estimate the number of underlying facies in a set of drill cuttings based on their micro-CT images. The basic idea behind GMMs is to model the distribution of the data as a mixture of several Gaussian distributions, where each component represents a distinct underlying facies.

To apply GMMs to the problem of grouping drill cuttings, the one or more processors first extract relevant features from the micro-CT images using techniques described above. These features may then be used as input to the GMM procedure, which estimates the parameters of the underlying Gaussian mixture model based on the observed data. One of the advantages of GMMs is that they can estimate the number of components in the mixture model automatically, without requiring prior knowledge of the number of facies. This may be achieved by using information criteria, such as the Bayesian Information Criterion (BIC) or the Akaike Information Criterion (AIC), which are statistical measures of the goodness of fit of a model and penalize models with more components to avoid overfitting.

Once the GMM model is trained, it can be used to predict the most likely facies for each individual drill cutting based on the posterior probabilities of belonging to each component. This may be performed using maximum a posteriori (MAP) estimation. One limitation of GMMs, however, is that they assume that the underlying facies are Gaussian distributions, which may not always be the case in practice. Therefore, the one or more processors may also explore (e.g., simultaneous explore) both k-means clustering and GMM approaches to reach a definite conclusion on the number of facies.

Certain aspects of the present disclosure provide techniques for mapping drill cuttings to cores. For example, when there are core plugs available from a geological reservoir, there may be some interest in finding the correlation between those plugs and the drill cuttings that were acquired from the same reservoir. Accordingly, techniques for mapping the drill cuttings to the facies exemplified in the cores may be useful, especially for calibration and validation studies in the lab, and for use in DRT. Techniques described herein may be implemented by one or more processors to find this information.

In some cases, to map the identified drill cuttings to corresponding core plugs, one or more processors may act according to the following steps: First, the one or more processors obtain high-resolution 3D images of the core plugs using micro-CT scanning and pre-process and segment the images to obtain trinary and binary image datasets for each core plug. The one or more processors then modify image resolution using bi-cubic interpolation such that all rock and drill cutting samples have the same resolution. The processors use the same feature extraction techniques used for the drill cuttings to extract features from the binary image datasets of the core plugs. Next, the one or more processors apply similar clustering procedure as those described above to cluster the core plugs based on their features. This may group the core plugs into different classes that may correspond to different facies. The processors then apply the same clustering algorithms to drill cuttings to group them into different classes that may correspond to different facies.

Once the number clusters for core plugs and drill cuttings are obtained, they are compared against each other; whichever case has a larger number of clusters or facies represents the reservoir in a more refined and inclusive manner. The trained clustering procedure with higher number of clusters is applied to feature space of the other case (e.g., plugs vs. cuttings) to infer the refined facies to which they belong. These steps are repeated on a combined set of feature vectors from both plugs and cuttings to ensure the accuracy of the inference.

Next, the one or more processors compute the mean feature vector for each cluster of core plugs and compare it to the feature vectors of the drill cuttings to determine the closest match and further ensure the accuracy of mapping between plugs and cuttings. If the processors are able to generate a valid correlation map, the processor update the sampling of cuttings, and the steps above are repeated until a similarity map is established between the core plugs and the new drill cuttings. In some cases, the one or more processors may check the validity of the obtained results against any available and reliable geological or well log information.

Aspects described herein provide an automated approach for classifying drill cuttings into different facies based on micro-CT high-resolution 3D images without the need for manual labeling or prior knowledge of the number of facies. One or more processors may use feature extraction techniques such as PCA or auto-encoders to capture the underlying patterns and variations in the images and clustering procedures such as GMMs or k-means to group the samples into different classes that represent the underlying facies.

### Aspects Related to Segmentation Workflow

According to aspects of the present disclosure, one or more processors may be configured to conduct pore-scale primary drainage and waterflooding in digital representations of reservoir cores generated from drill cuttings. To this end, the processors first carry out statistical sampling and preliminary screening of the drill cuttings based on their physical attributes. The processors may then clean and condition the selected drill cuttings and generate a set of corresponding 3D images. The processors may then process and segment the 3D images and subsequently feed the segmented images into an artificial intelligence (AI)-assisted image processing platform to cluster and map the drill cuttings to a rock facies (e.g., targeted rock facies or randomized facies) based on certain properties of the drill cuttings (e.g., petrophysical characteristics). Next, the processors perform pore network extraction and generation to stochastically generate ensemble of digital replicates of the drill cuttings using representative pore networks extracted from segmented micro-CT images of drill cuttings. Finally, the processors perform multiphase dynamic flow and transport simulations in the digital replicates to predict the macroscopic flow and transport properties of the rock facies that correspond to the selected drill cuttings.

Aspects of the present disclosure describe an image processing platform for multi-component (e.g., multi-mineral rock) image analysis using an artificial intelligence (AI)-assisted approach with automated GPU-accelerated workflows. Aspects of the present disclosure provide techniques for processing images obtained by a micro-CT scanner in the lab, and rendering and processing image datasets to obtaining reliable digital replicates that are in one-to-one correspondence to the porous media samples (e.g., studied in the laboratory). Aspects of the present disclosure further provide techniques to visualize and massively process raw 3D images of a wide range of porous media materials while using the computational power of multi-core, multi-GPU systems through domain decomposition techniques.

The image processing platform may include preprocessing and image enhancement, which may use an efficient variant of nonlocal means filtering accelerated on one or more GPUs, followed by a segmentation workflow. The segmentation workflow may consist of two major components. The first component may configure one or more CPUs and/or one or more GPUs to perform adaptive local thresholding, automated global thresholding, top-hat transformation of raw image, and several morphological operations to obtain a conservative estimate of macro-pores as one segment in the raw micro-CT image of the porous media sample (e.g., a rock sample). Next, the one or more CPUs and/or the one or more GPUs may perform GPU-accelerated clustering on an image masked by the one or more CPUs to find the segments for grains within the porous media sample having a certain composition (e.g., minerals like clay that can host micro-porosity but are often not resolved by the imaging resolution). This clustering-based multi-mineral segmentation of porous media may include training a model based on a portion of pixels in each image slice. The trained model may be used to predict the cluster centroids to which each pixel of the image belongs. After applying the trained model, a merging process may take place to merge like components of the porous media sample. For example, detected minerals may be merged into a more representative ‘grain’ cluster and the micro-porosity-containing minerals into an intermediate ‘clay’ phase. A three-class segmentation may be obtained, and the corresponding three-class markers may be generated following automated workflows, which may conservatively represent ‘definite’ classes (e.g., pores, clays, and grains). The first component of the platform may be performed for all the slices of a 3D stack in parallel using multi-core CPU threads as well as multi-GPUs at the same time. This may facilitate increased processing speeds.

In the second component of the workflow, a variant of marker-controlled 3D watershed segmentation may be performed. The watershed segmentation employs a compactness parameter to regularize the regional seed growth. Due to the extremely high computational load of performing 3D watershed-based segmentation for the entire stack, a parallel version may be implemented to simultaneously perform the segmentation on multiple partially overlapping subvolumes with certain subvolume and pad sizes determined a priori to eliminate the end effect at the margins of each vertical subvolume. This approach may significantly reduce computational time for a typical gray-scale image stack (e.g., containing 2000 slices with each slice comprising about 9 million pixels). Marker-controlled watershed segmentation may allow full connectivity to be maintained in 3D space for each of the defined classes.

Aspects of the present disclosure provide techniques for emulating physical properties (e.g., petrophysical properties) of an input pore network to mimic certain physical structures by replicating the support of its pore-bodies. Specifically, existing pore-bodies may be sampled from the input network and randomly placed within the desired dimensions of the generated pore network. Then, using the connectivity information of the input pore-body, several layers of surrounding pore-elements may be added to the generated network. In this way, the local topology of the input network may be preserved within the generated pore network. In certain cases, the placement of each pore-body and the replication of their support can be performed independently with limited communication between threads. After the generated pore network has an equivalent density of pore bodies as the input network, additional pore throats may be added to provide connections between each replicated support. Placement of these pore throats is determined using the coordination numbers of the input network, and the geometrical information of the neighboring pore throats of each pore-body.

Additionally, pore network extraction performed by processors operating in sequence may be unable to support the computational load associated with pore networks. Techniques implemented to address issues with resolution sufficiency may also address issues arising from the computational expense of high-resolution imaging.

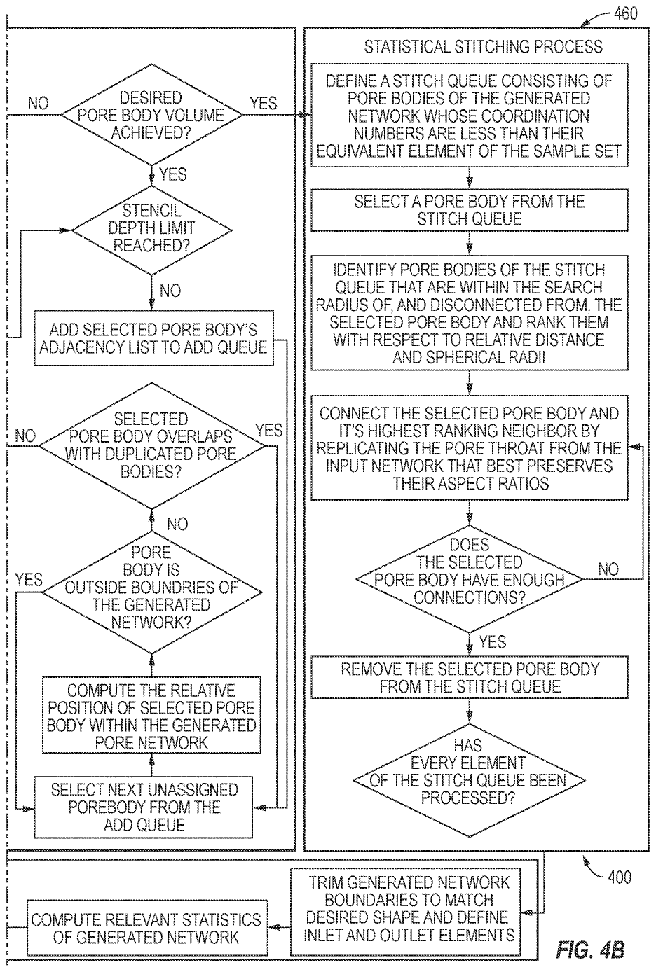

A flow chart of a stencil-based pore network generation process 400 is provided in FIG. 4. According to certain aspects, construction of a generated pore network consists of four major steps performed by one or more processors: pre-processing 410, stochastic pore network stencil replication 430, statistical stitching process 460, and post-processing 480. During the pre-processing step 410 is performed to read in and processing the representative pore network to define the sample set. During stochastic pore network stencil replication 430, the elements of the sample set are randomly distributed within the desired dimensions of the generated pore network. After each sample element is placed a depth-limited breadth first search is used to identify and replicate its stencil elements. In the next step, a statistical stitching process 460 is used to add pore throats and define connections between the duplicated stencils of the generated pore network. In this step, if the coordination number a duplicated pore body is less than its corresponding value in the sample set then pore throats may be assigned between it and its highest-ranking neighbors. These added pore throats may be chosen so that they preserve the statistics of the representative pore network. Finally, the resulting pore network undergoes post-processing 480 to match the desired geometry, define inlet and outlet pore elements, and is adjusted to match the porosity data of the representative pore network. The procedure described herein replicates the representative pore network and, in this sense, is constructed independently of the porous medium we wish to emulate.

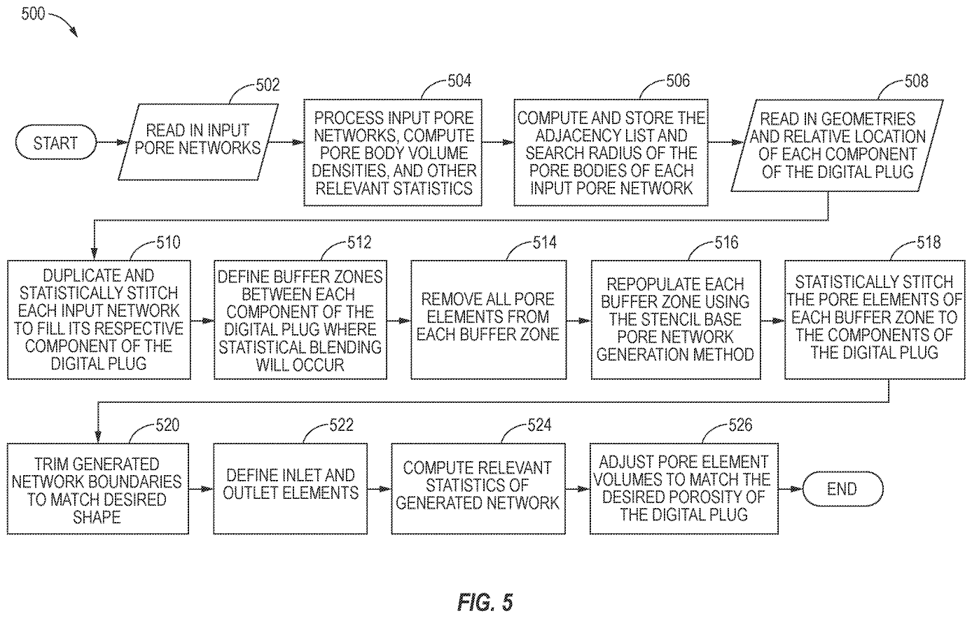

A pore network duplication and blending procedure 500 that may be used to construct a representation of a porous media sample is illustrated in FIG. 5. Aspects of the present disclosure provide techniques for pore network duplication and blending. One or more processors (e.g., one or more CPUs) may perform pore network duplication and blending procedures to generate a large-scale representation of a porous media sample (e.g., a digital plug). The one or more processors perform a pre-processing of the representative pore networks, local pore network duplication, stochastic stencil-based pore network blending, and post-processing of the digital plug. At operation 502, input pore networks are read into the one or more processors. At operation 504, the input pore networks are processing, the pore body volume densities are computed, and other relevant statistics are processed; at operation 506 an adjacency list and search radius of each pore body of each input pore network is computed and stored; and at operation 508, the geometries and relative locations of each component of the digital plug are read in. At operation 510, during the local pore network duplication step, the processors may duplicate representative pore networks to fill their respective volumes within the digital plug and then stitch the representative pore networks using a statistical stitching process. In another example, if the samples are sufficiently homogeneous, a stochastic stencil-based pore network generation routine may be used in place of this pore network duplication step. At operation 512, one or more buffer zones between each component of the digital plug, where the statistical blending will occur, are defined. At operation 514, all pore elements from each of the one or more buffer zones are removed. At operation 516, the one or more processors may employ a stencil-based pore network generation method to blend each component of the heterogeneous digital plug. At operation 518, the pore elements of each of the one or more buffer zones are stitched to the components of the digital plug. Finally, the resulting pore network is post-processed. The post-processing includes an operation 520 to trim generated network boundaries to match the desired geometry, an operation 522 to define inlet and outlet pore elements, an operation 524 to compute relevant statistics of the generated network, and an operation 526 to adjust to match the desired porosity data.

Where pore network duplication and blending is applied to drill cuttings, the volumes within the digital plug are discretized and assigned representative drill cuttings. When extracting a porous media sample from drill cuttings, the processors will need to account for the lack of coordinates associated with each drill cutting. The lack of coordinates for each drill cutting raise an additional issue of placement of the drill cuttings within the digital plug. Because placement of drill cutting information within the digital plug cannot be based on an actual coordinates associated with the porous media, one or more processors grab a random point in space inside of the discretization and define that point as a seed to generate a digital facies associated with that cutting. Multiple seed points may be generated to create a digital facies map that is able to reflect the petrochemical properties of the drill cuttings. In some cases, the processors may apply an inflation step to each seed point to fill the volume. During the inflation step, the processors may randomly assign volumes for each drill cutting to fill the digital plug. After the inflation step, the processors may define interfaces between each drill cutting volume within the digital plug and resolve those interfacing according to the methods described above.

Aspects of the present disclosure provide an efficient and robust framework to carry out pore-scale displacements based on the fluid pressure fields that are updated frequently during flow procedures. The efficiency of framework combined with the parallelization of the platform across one or more CPUs may facilitate performing fluid flow prediction in large core-sized pore networks within a practical amount of time.

Aspects of the present disclosure provide an efficient and robust dynamic pore-network modeling framework to carry out pore-scale displacements based on the fluid pressure fields that are updated frequently during flow simulations. The efficiency of framework combined with the parallelization of the platform across one or more CPUs may facilitate performing fluid flow prediction in large core-sized pore networks within a practical amount of time. This framework is capable of modeling two-phase flow under complex wettability and fluid flow conditions by explicitly accounting for the capillary, viscous, and buoyance forces.

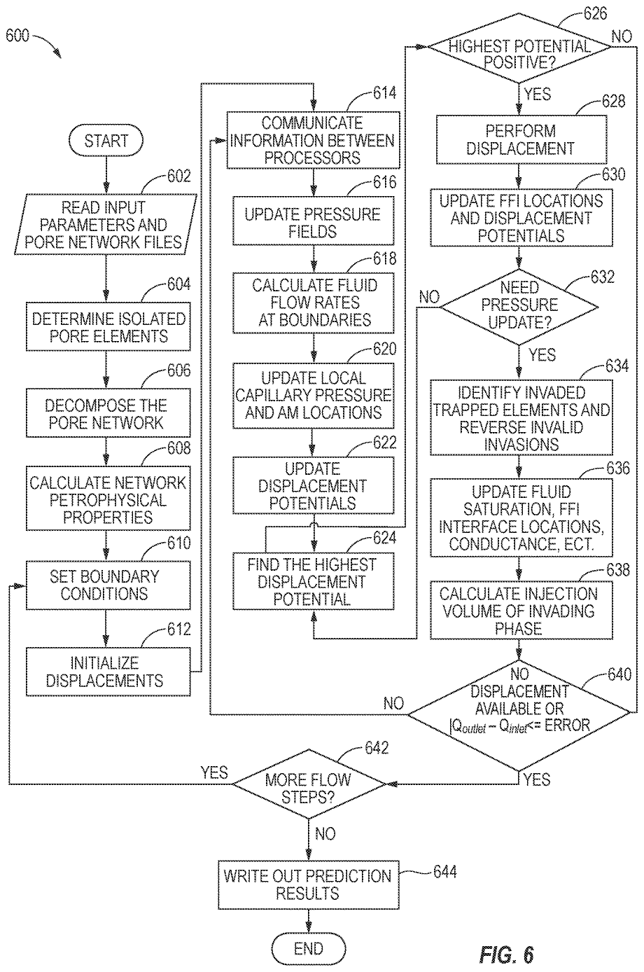

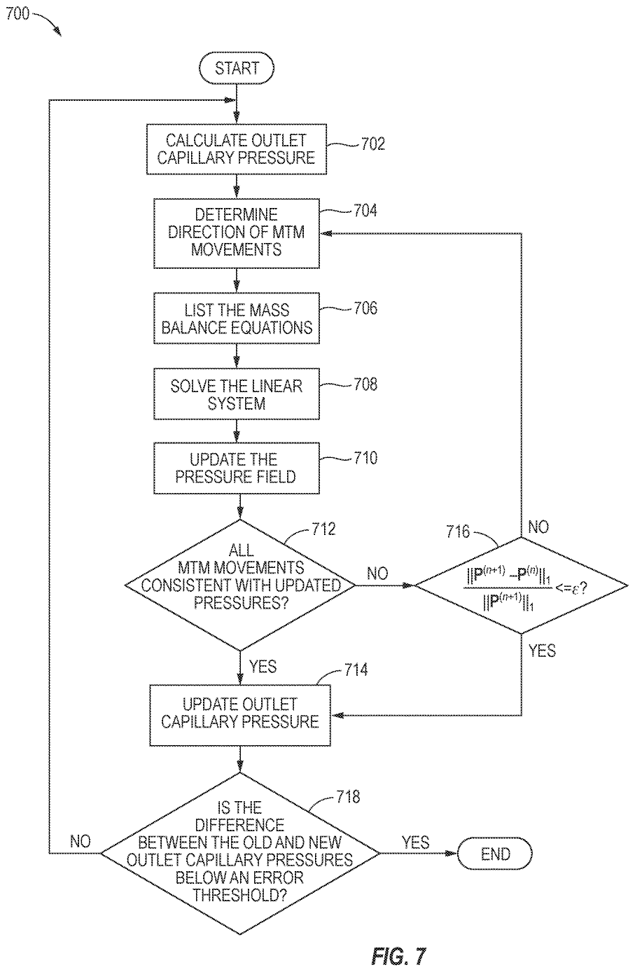

According to certain aspects, FIG. 6 depicts an example flow diagram describing example actions taken by one or more CPUs to implement a fluid flow prediction process 600. At operation 602, one or more CPUs read in one or more input parameters and pore network files. For example, the CPUs may obtain an extracted pore network model at the start of prediction. At operation 604 and 606, the CPUs then determine isolated pore elements and decompose the network. Network decomposition may allow the one or more CPUs to process the fluid flow prediction more efficiently. At operation 608, 610, and 612, each of the CPUs calculates petrophysical properties of the dynamic pore network modeling (DPNM), set boundary conditions, then initializes displacements, respectfully. At operation 614, the information calculated in operations 608, 610, and 612 is communicated between CPUs. At operation 616, the CPUs then begin the process of updating pressure fields within the DPNM (discussed below with respect to FIG. 7). At operation 618, 620, and 622, the CPUs calculate the fluid rates at boundaries, update local capillary pressure and arc meniscus (AM) locations, and update displacement potentials, respectively. At operation 624, the CPUs determine the highest displacement potential. If the highest displacement potential is positive, the fluid flow prediction process 600 proceeds to operation 628. At operation 628, the CPUs perform the displacement associated with the potential. If the highest positive displacement potential is not positive, the fluid flow prediction process 600 proceed to operation 640 and to operation 642. At operation 642, the CPUs determine whether there are more flow steps. If yes, the fluid flow prediction process 600 returns to operation 610. If no, the fluid flow prediction process 600 proceed to operation 644. At operation 644, the CPUs write our prediction results and end the fluid flow prediction.

After the CPUs perform a displacement at operation 628, the fluid flow prediction process 600 proceeds to operation 630. At operation 630, the CPUs update fluid-fluid interface (FFI) locations and displacement potentials. The CPUs then determine whether the displacement requires a pressure displacement. If the displacement requires a pressure update, the fluid flow prediction process 600 proceeds to operation 634, 636, 638 and 640. At operations 634, 636, 638, and 640, the CPUs identify invaded trapped elements and reverse the invalid invasions, CPUs update fluid saturation, FFI locations, conductance, and other parameters, calculate the injection volume of the invading phase, and evaluate whether there is a displacement available or whether the difference between the outlet and inlet capillary pressures is lower than a threshold error value, respectively. If the displacement does not require a pressure update, the fluid flow prediction process 600 returns to operation 624.

At operation 642, if there is no displacement available or the difference is lower than the threshold error value, the fluid flow prediction process 600 proceeds to operation 642. At operation 642, the CPUs determine whether there are more flow steps. If there are more flow steps, then the fluid flow prediction process 600 returns to operation 610. If there are not more flow steps, the fluid flow prediction process 600 proceeds to operation 644. If the displacement has a difference is greater than the threshold error value, the fluid flow prediction process 600 returns to operation 614.

FIG. 7 depicts an example flow diagram describing example actions taken by one or more CPUs to implement fluid flow prediction, specifically with respect to the updating fluid pressure fields action described in FIG. 6. At operation 702 of a pressure field update process 700, one or more CPUs calculate outlet capillary pressure. At operation 704, based on the fluid pressure fields from previous step, the CPUs determine the direction of main-terminal meniscus (MTM) movements. At operation 706, the CPUs then list the mass balance equations for each pore element accounted for within the fluid flow prediction procedure. At operation 708 and 710, the CPUs solve the linear system for the mass balance equations and update the pressure field, respectively. At operation 712, the CPUs determine whether all MTM movements are consistent with the updated pressure fields. If MTM movements are not consistent with the updated pressure fields, the pressure field update process 700 proceeds to operation 716. At operation 716, the CPUs determine whether the relative error of the vector of the pressure values calculated over two consecutive iterations satisfies an error tolerance. If the error tolerance is satisfied, the pressure field update process 700 proceeds to operation 714. If the error tolerance is not satisfied, the pressure field update process 700 returns to operation 704 to repeat the determination of MTM movements and proceeding steps until the difference between the previous and current pressure updates divided by the current pressure update is less than a threshold error value is less than a threshold error value. At operation 714, the CPUs updates the outlet capillary pressure. After updating the outlet capillary pressure, the CPUs determine whether the difference between the newly computed and previous outlet capillary pressures is below a certain threshold. If the difference is not below a certain threshold, the pressure field update process 700 returns to operation 702. The CPUs start over, calculating the outlet capillary pressure again. If the difference is below a certain threshold, the pressure field update process 700 ends. The CPUs end the pressure update and output the outlet capillary pressure for use in the fluid flow prediction procedure of FIG. 7.

According to certain aspects of the present disclosure, the CPUs may use simplifying assumptions specific to the needs of dynamic pore-network modeling of fluid flow through mixed-wet porous media. In one case, all pore bodies and pore throats are assumed to possess non-zero values for volume, conductance, and local capillary pressure. In one case, the CPUs may assume any commonly used cross-sectional shapes for each pore element within the pore network model, including circle, square, and irregular triangle cross-sections.

### Example Methods

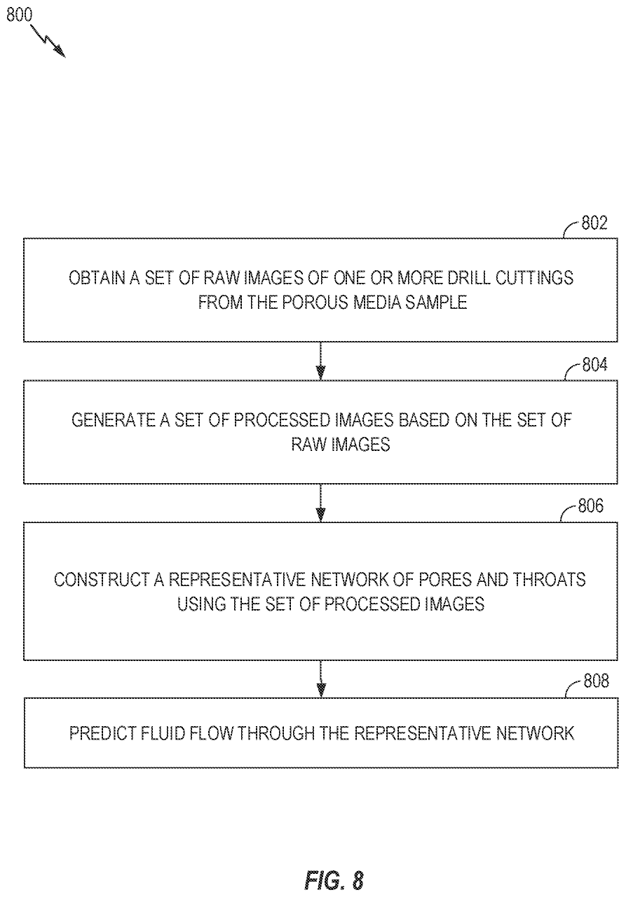

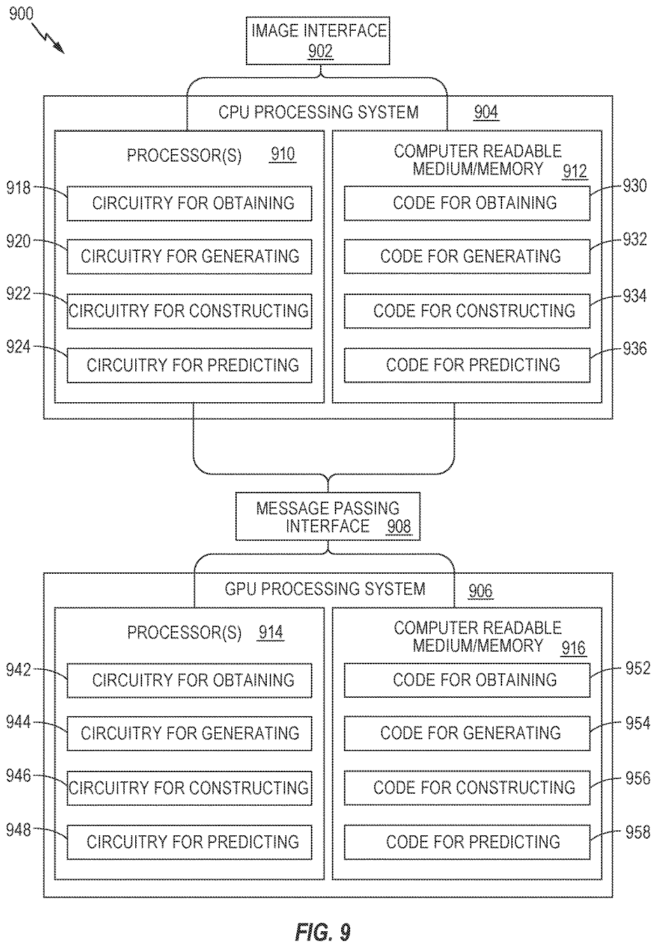

FIG. 8 depicts a method 800 for processing images of porous media by one or more CPUs, such as the CPUs of the device 900 of FIG. 9.

Method 800 begins at 802 with one or more CPUs obtaining a set of raw images of one or more drill cuttings from the porous media sample.

Method 800 continues to step 804 with one or more CPUs generating a set of processed images based on the set of raw images.

Method 800 continues to step 806 with one or more CPUs constructing a representative network of pores and throats using the set of processed images.

Method 800 continues to step 808 with one or more CPUs predicting fluid flow through the representative network In one aspect, method 800, or any aspect related to it, may be performed by an apparatus, such as imaging device 800 of FIG. 8, which includes various components operable, configured, or adapted to perform the method 800. Device 900 is described below in further detail.

Note that FIG. 8 is just one example of a method, and other methods including fewer, additional, or alternative steps are possible consistent with this disclosure.

### Example Device

FIG. 9 depicts aspects of an example porous media device 900. In some aspect, the device 900 comprises one or more CPUs, one or more GPUs, or both as described above with respect to FIG. 8.

The device 900 includes a CPU processing system 904 coupled to an image interface 902 (e.g., a user interface or and/or an image generator such as a commercial micro-CT scanner). The CPU processing system 904 may be configured to perform processing functions for the device 900, including multiscale imaging of porous media generated by the device 900.

The CPU processing system 904 includes one or more processors 910. The one or more processors 910 are coupled to a computer-readable medium/memory 912 via a bus. The one or more processors 910 and the computer-readable medium/memory 912 may communicate with the one or more processor 914 and the computer-readable medium/memory 916 of the GPU processing system 906 via a message passing interface (MPI) 908. In certain aspects, the computer-readable medium/memory 912 is configured to store instructions (e.g., computer-executable code) that when executed by the one or more processors 910, cause the one or more processors 910 to perform the method 800 described with respect to FIG. 8, or any aspect related to it. Note that reference to a processor performing a function of device 900 may include one or more processors performing that function of device 900.

In the depicted example, computer-readable medium/memory 912 stores code (e.g., executable instructions) for obtaining 930, code for generating 932, code for constructing 934, and code for predicting 936. Processing of the code 930-936 may cause the imaging device 900 to perform the method 2700 described with respect to FIG. 8, or any aspect related to it.

The one or more processors 910 include circuitry configured to implement (e.g., execute) the code stored in the computer-readable medium/memory 912, including circuitry for obtaining 918, circuitry for generating 920, circuitry for constructing 922, and circuitry for predicting 924. Processing with circuitry 918-924 may cause the imaging device 900 to perform the method 800 described with respect to FIG. 8, or any aspect related to it.

Various components of the device 900 may provide means for performing the method 800 described with respect to FIG. 8, or any aspect related to it. Various components of the device 900 may provide means for performing the method 800 described with respect to FIG. 8, or any aspect related to it.

The device 900 includes a GPU processing system 906. The GPU processing system 906 may be configured to perform processing functions for the device 900, including multiscale imaging of porous media generated by the device 900.

The GPU processing system 906 includes one or more processors 914. The one or more processors 914 are coupled to a computer-readable medium/memory 916 via a bus. The one or more processors 914 and the computer-readable medium/memory 916 may communicate with the one or more processor 910 and the computer-readable medium/memory 912 of the CPU processing system 904 via an MPI 908. In certain aspects, the computer-readable medium/memory 216 is configured to store instructions (e.g., computer-executable code) that when executed by the one or more processors 914, cause the one or more processors 914 to perform the method 800 described with respect to FIG. 8, or any aspect related to it. Note that reference to a processor performing a function of device 900 may include one or more processors performing that function of device 900.

In the depicted example, computer-readable medium/memory 916 stores code (e.g., executable instructions) for obtaining 952, code for generating 954, code for constructing 956, code for predicting 958. Processing of the code 952-958 may cause the imaging device 900 to perform the method 2800 described with respect to FIG. 8, or any aspect related to it.

The one or more processors 914 include circuitry configured to implement (e.g., execute) the code stored in the computer-readable medium/memory 916, including circuitry for obtaining 942, circuitry for generating 944, circuitry for constructing 946, and circuitry for predicting 948. Processing with circuitry 942-948 may cause the imaging device 900 to perform the method 800 described with respect to FIG. 8, or any aspect related to it.

Various components of the imaging device 900 may provide means for performing the method 800 described with respect to FIG. 8, or any aspect related to it.

### Example Aspects

Implementation examples are described in the following numbered clauses:

Aspect 1: A method for processing a porous media sample by one or more processing units, comprising: obtaining a set of raw images of one or more drill cuttings from the porous media sample; generating a set of processed images based on the set of raw images; constructing a representative network of pores and throats using the set of processed images; and predicting fluid flow through the representative network.

Aspect 2: The method of aspect 1, wherein the porous media sample is a rock sample.

Aspect 3: A non-transitory computer-readable medium comprising computer-executable instructions that, when executed by one or more processors, cause one or more processing units to: obtain a set of raw images of one or more drill cuttings from the porous media sample; generate a set of processed images based on the set of raw images; construct a representative network of pores and throats using the set of processed images; and predict fluid flow through the representative network.

Aspect 4: The method of aspect 3, wherein the porous media sample is a rock sample.

Aspect 5: An apparatus, comprising: a memory comprising executable instructions; and a processor configured to execute the executable instructions and cause the apparatus to perform a method in accordance with any one of Aspects 1-4.

Aspect 6: An apparatus, comprising means for performing a method in accordance with any one of Aspects 1-4.

Aspect 7: A non-transitory computer-readable medium comprising executable instructions that, when executed by a processor of an apparatus, cause the apparatus to perform a method in accordance with any one of Aspects 1-4.

Aspect 8: A computer program product embodied on a computer-readable storage medium comprising code for performing a method in accordance with any one of Aspects 1-4.

### Additional Considerations

The preceding description is provided to enable any person skilled in the art to practice the various aspects described herein. The examples discussed herein are not limiting of the scope, applicability, or aspects set forth in the claims. Various modifications to these aspects will be readily apparent to those skilled in the art, and the general principles defined herein may be applied to other aspects. For example, changes may be made in the function and arrangement of elements discussed without departing from the scope of the disclosure. Various examples may omit, substitute, or add various procedures or components as appropriate. For instance, the methods described may be performed in an order different from that described, and various actions may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing, decomposing, and the like.

The methods disclosed herein comprise one or more operations or actions for achieving the methods. The method operations and/or actions may be interchanged with one another without departing from the scope of the claims. In other words, unless a specific order of operations or actions is specified, the order and/or use of specific operations and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in the Figures, those operations may have corresponding counterpart means-plus-function components with similar numbering.

The following claims are not intended to be limited to the aspects shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for processing a porous media sample by one or more processing units, comprising:
obtaining a set of raw images of one or more drill cuttings from the porous media sample;
generating a set of processed images based on the set of raw images;
constructing a representative network of pores and throats using the set of processed images; and
predicting fluid flow through the representative network.

2. The method of claim 1, wherein the porous media sample is a rock sample.

3. The method of claim 1, wherein predicting fluid flow comprises:
calculating capillary pressure of an outlet of the representative network;
determining a main-terminal meniscus (MTM) movement from the capillary pressure;
listing the mass balance equations for fluid phases inside the network of pores and throats;
solving the linear system for the mass balance equations;
updating a pressure field;
determining if an updated pressure field is consistent with the MTM movement;
repeating the updating of the pressure field; and the determining if an updated pressure field is consistent with the MTM movement upon determining the MTM movements are not consistent with the updated pressure fields;
determining whether the relative error of a vector of pressure field satisfies an error tolerance;
repeating determining a main-terminal meniscus (MTM) movement from the capillary pressure; listing the mass balance equations for fluid phases inside the network of pores and throats; solving the linear system for the mass balance equations; updating a pressure field;
determining if an updated pressure field is consistent with the MTM movement; and
determining whether the relative error of a vector of pressure field satisfies an error tolerance upon determining the relative error does not satisfy the error tolerance;
updating the outlet capillary pressure; and
determining whether the difference between an updated outlet capillary pressure and outlet capillary pressure is below a threshold.

4. The method of claim 1, wherein predicting fluid flow comprises:
reading in one or more parameters and pore network files;
determining isolated pore elements and decomposing the pore network;
calculating petrophysical properties, setting the boundary conditions, and initializing displacements of the dynamic pore network modeling (DPNM);
updating fluid pressure fields within the pore network;
calculating a fluid rate at the boundaries, updating local capillary pressures and arc meniscus (AM) locations, and updating displacement potentials;
determining a highest displacement potential is positive;
performing a displacement associated with the highest displacement potential upon determining that the highest displacement potential is positive;
updating the fluid-fluid interface (FFI) locations and displacement potentials;
determining if the displacement requires a pressure displacement; and
identifying invaded trapped elements; reversing invalid invasions; updating fluid saturation, FFI locations, conductance, and other parameter; calculating the injection volume of an invading phase; and evaluating a whether there is a displacement available or whether the difference between an outlet flow rate and inlet flow rate is lower than a threshold error value, upon determining the displacement requires a pressure update.

5. The method of claim 1, further comprising:
trimming representative network boundaries to match desired shape and to define an inlet element and an outlet element;
computing relevant statistics of representative network;
adjusting a pore element volume to match the porosity of the representative network.

6. A non-transitory computer-readable medium comprising computer-executable instructions that, when executed by one or more processors, cause one or more processing units to:
obtain a set of raw images of one or more drill cuttings from the porous media sample;
generate a set of processed images based on the set of raw images;
construct a representative network of pores and throats using the set of processed images; and
predict two-phase fluid flow through the representative network.

7. The method of claim 3, wherein the porous media sample is a rock sample.

8. The method of claim 3, wherein predicting fluid flow comprises:
calculating capillary pressure of an outlet of the representative network;
determining a main-terminal meniscus (MTM) movement from the capillary pressure;
listing the mass balance equations for fluid phases inside the network of pores and throats;
solving the linear system for the mass balance equations;
updating a pressure field;
determining if an updated pressure field is consistent with the MTM movement;
repeating the updating of the pressure field upon determining the MTM movements are not consistent with the updated pressure fields;
determining whether the relative error of a vector of pressure field satisfies an error tolerance;
repeating determining a main-terminal meniscus (MTM) movement from the capillary pressure; listing the mass balance equations for fluid phases inside the network of pores and throats; solving the linear system for the mass balance equations; updating a pressure field;
determining if an updated pressure field is consistent with the MTM movement; and
determining whether the relative error of a vector of pressure field satisfies an error tolerance upon determining the relative error does not satisfy the error tolerance;
updating the outlet capillary pressure; and
determining whether the difference between an updated outlet capillary pressure and outlet capillary pressure is below a threshold.

9. The method of claim 3, wherein predicting fluid flow comprises:
reading in one or more parameters and pore network files;
determining isolated pore elements and decomposing the pore network;
calculating petrophysical properties, setting the boundary conditions, and initializing displacements of the dynamic pore network modeling (DPNM);
updating fluid pressure fields within the pore network;
calculating a fluid rate at the boundaries, updating a local capillary pressure and arc meniscus (AM) locations, and updating a displacement potentials;
determining a highest displacement potential is positive;
performing a displacement associated with the highest displacement potential upon determining that the highest displacement potential is positive;
updating the fluid-fluid interface (FFI) locations and displacement potentials;
determining if the displacement requires a pressure displacement;
identifying invaded trapped elements; reversing invalid invasions; updating fluid saturation, FFI locations, conductance, and other parameters; calculating the injection volume of an invading phase; and evaluating a whether there is a displacement available or whether the difference between an outlet flow rate and inlet flow rate is lower than a threshold error value, upon determining the displacement requires a pressure update.

10. The method of claim 3, further comprising:
trimming representative network boundaries to match desired shape and to define an inlet element and an outlet element;
computing relevant statistics of representative network;
adjusting a pore element volume to match the porosity of the representative network.

11. An apparatus for processing a porous media sample comprising a memory and one or more processors, the one or more processors are configured to cause the apparatus to predict fluid flow by:
obtaining a set of raw images of one or more drill cuttings from the porous media sample;
generating a set of processed images based on the set of raw images;
constructing a representative network of pores and throats using the set of processed images; and
predicting fluid flow through the representative network.

12. The apparatus of claim 5, wherein the porous media sample is a rock sample.

13. The apparatus of claim 5, wherein the one or more processors are configured to predict fluid flow by:
calculating capillary pressure of an outlet of the representative network;
determining a main-terminal meniscus (MTM) movement from the capillary pressure;
listing the mass balance equations fluid phases inside for the network of pores and throats;
solving the linear system for the mass balance equations;
updating a pressure field;
determining if an updated pressure field is consistent with the MTM movement;
repeating the updating of the pressure field upon determining the MTM movements are not consistent with the updated pressure fields;
determining whether the relative error of a vector of pressure field satisfies an error tolerance;
repeating determining a main-terminal meniscus (MTM) movement from the capillary pressure; listing the mass balance equations for fluid phases inside the network of pores and throats; solving the linear system for the mass balance equations; updating a pressure field;
determining if an updated pressure field is consistent with the MTM movement; and
determining whether the relative error of a vector of pressure field satisfies an error tolerance upon determining the relative error does not satisfy the error tolerance;
updating the outlet capillary pressure; and
determining whether the difference between an updated outlet capillary pressure and outlet capillary pressure is below a threshold.

14. The apparatus of claim 5, wherein the one or more processors are configured to cause the apparatus to predict fluid flow by:
reading in one or more parameters and pore network files;
determining isolated pore elements and decomposing the pore network;
calculating petrophysical properties, setting the boundary conditions, and initializing displacements of the dynamic pore network modeling (DPNM);
updating fluid pressure fields within the pore network;
calculating a fluid rate at the boundaries, updating local capillary pressures and arc meniscus (AM) locations, and updating displacement potentials;
determining a highest displacement potential is positive;
performing a displacement associated with the highest displacement potential upon determining that the highest displacement potential is positive;
updating the fluid-fluid interface (FFI) locations and displacement potentials;
determining if the displacement requires a pressure update; and
identifying invaded trapped elements; reversing invalid invasions; updating fluid saturation, FFI locations, conductance, and other parameters; calculating the injection volume of an invading phase; and evaluating a whether there is a displacement available or whether the difference between an outlet flow rate and inlet flow rate is lower than a threshold error value, upon determining the displacement requires a pressure updates.

15. The apparatus of claim 5, further comprising the one or more processors are configured to cause the apparatus to:
trim representative network boundaries to match desired shape and to define an inlet element and an outlet element;
compute relevant statistics of representative network;
adjust a pore element volume to match the porosity of the representative network.
